(19) United States
(12) Patent Application Publication
(10) Pub. No.: US 2014/0209501 A1
(43) Pub. Date: Jul. 31, 2014

(54) Methods and Systems for Storing Medical Implants Under Sustained Vacuum

(71) Applicant: Alphatec Spine, Inc., Carlsbad, CA (US)

(72) Inventors: Amit GOVIL, San Diego, CA (US); Christian Gabriel GAMBOA, San Diego, CA (US); Neil Irvin THOMPSON, San Marcos, CA (US); Jeffrey Allen GUYER, San Diego, CA (US); Markanthony B. FLORES, San Diego, CA (US); Sudhanshu SOMASUNDAR, San Diego (IN)

(73) Assignee: Alphatec Spine, Inc., Carlsbad, CA (US)

(21) Appl. No.: 14/242,270

(22) Filed: Apr. 1, 2014

Related U.S. Application Data
(63) Continuation-in-part of application No. 12/251,297, filed on Oct. 14, 2008, now Pat. No. 8,080,060.
Application No. 12/251,297 is a continuation-in-part of application No. 12/130,920, filed on May 30, 2008, now Pat. No. 8,070,740.
(60) Provisional application No. 60/932,479, filed on May 30, 2007.

(51) Int. Cl. A61F 2/00

(52) U.S. Cl. CPC A61F 2/0095
USPC 206/570

(57) ABSTRACT

Methods and systems for improving the shelf life of a medical graft generally comprising a medical implant container having at least one graft cavity configured to hold at least one graft under a first vacuum and a needle entry port in fluid communication with the at least one graft cavity, the needle entry port being configured to receive and communicate a material to the at least one graft cavity and an outer chamber configured to hold the medical implant container under a second vacuum.

### CROSS REFERENCE TO RELATED APPLICATIONS

This application is a continuation-in-part of U.S. application Ser. No. 12/251,297, filed on Oct. 14, 2008, entitled “PROCESSES AND SYSTEMS FOR LOADING MEDICAL IMPLANTS WITH SIMULATIVE GROWTH AGENTS”, which is a continuation-in-part of U.S. application Ser. No. 12/130,920, filed on May 30, 2008, entitled “PROCESSES AND SYSTEMS FOR HYDRATING AND SEEDING MEDICAL IMPLANTS WITH BIOLOGICAL COMPONENTS”, which claims priority to U.S. Provisional Application No. 60/932,479, filed May 30, 2007, entitled “PROCESSES AND SYSTEMS FOR HYDRATING AND SEEDING MEDICAL IMPLANTS WITH BIOLOGICAL COMPONENTS”, the contents of which are incorporated herein by reference in their entirety.

This application also claims priority to U.S. Provisional Application No. 61/119,688, filed on Dec. 3, 2008, entitled “PROCESSES AND SYSTEMS FOR LOADING MEDICAL IMPLANTS WITH SIMULATIVE GROWTH AGENTS” and U.S. Provisional Application No. 61/138,842, filed on Dec. 18, 2008, entitled “OUTER VACUUM CHAMBER TO INCREASE EFFICIENCY OF DRUG DELIVERY DEVICE”, the contents of which are incorporated herein by reference in their entirety.

### FIELD OF THE INVENTION

The present invention relates generally to medical implants and more particularly to methods and systems for storing medical implants under sustained vacuum.

### BACKGROUND

Bone grafting refers to a wide variety of medical and dental surgical procedures by which the formation of new bone in a patient is augmented or stimulated. Bone grafting is used in many types of orthopedic procedures to treat bone fractures or loss, to repair injured bone that has not healed, and to fuse together joints to prevent movement. With particular reference to the spine, grafts have been used to stabilize the spine and to prevent movement by selected vertebral segments, which may be a significant cause of pain in some patients. Grafts have also been used to correct or stop the progress of spinal deformity, such as scoliosis, and to provide structural support for fractures of the spine.

Suitable grafts can be harvested from bones in the patient's own body (autografts), from bones in members of the same species (allograft), and from bones in members of other animal species (xenograft). Alternatively, bone grafts can be created from a wide variety of natural and/or synthetic materials, such as collagen, polymers, hydroxyapatite, calcium sulfate, ceramics, and bioresorbable polymers, among many others. It is understood that bone grafts can include those which have a predetermined shaped or which are comprised of smaller particles that can be formed into a desired shape at the time of implantation.

Regardless of the source, bone grafts must be adequately preserved for later implantation in a surgical setting. One common practice is to dehydrate the grafts by freeze-drying. This not only extends the shelf-life of the bone grafts, it also inhibits bacterial growth within the graft. Before implanting the graft into a recipient, however, the graft must be reconstituted or rehydrated with a suitable liquid. This can be done by immersing the bone graft in the liquid. The problem with this approach, however, is that infusion of the liquid through the pores of the graft is typically unacceptably slow for a surgical environment and does not ensure thorough and complete infusion of the liquid throughout the graft. Moreover, this approach increases the likelihood of exposing the graft to environmental pathogens.

It would be desirable to develop a system and process for extending the shelf-life of vacuum on medical implants or grafts, particularly bone grafts.

### SUMMARY OF THE INVENTION

Methods and systems are disclosed herein storing medical implants under sustained vacuum.

In a first aspect, embodiments of the present invention provide a kit for storing medical grafts under vacuum, the kit comprising medical implant container having at least one graft cavity configured to hold at least one graft under a first vacuum and a needle entry port in fluid communication with the at least one graft cavity, the needle entry port being configured to receive and communicate a material to the at least one graft cavity, and an outer chamber configured to hold the medical implant container under a second vacuum.

In another aspect, embodiments of the present invention provide a medical graft storage system comprising at least one medical graft configured for implantation in a body, a medical implant container having at least one graft cavity configured to hold at least one graft under a first vacuum and a needle entry port in fluid communication with the at least one graft cavity, the needle entry port being configured to receive and communicate a material to the at least one graft cavity, and an outer chamber configured to hold the medical implant container under a second vacuum.

In another aspect, embodiments of the present invention provide a medical graft storage system comprising at least one medical graft configured for implantation in a body, a medical implant container having at least one graft cavity configured to hold at least one graft under a first vacuum and a needle entry port in fluid communication with the at least one graft cavity, the needle entry port being configured to receive and communicate a material to the at least one graft cavity, and an outer chamber configured to hold the medical implant container under a second vacuum.

In many embodiments, the first vacuum and second vacuum between 1 and 30 inHg.

In many embodiments, the first vacuum and second vacuum are substantially the same.

In many embodiments, the first vacuum and second vacuum are not substantially the same.

In many embodiments, the outer chamber is made of a material selected from the group consisting of SiOx, foil, ACLAR, EVOH, PVOH. Alox , SiOx-F and PET, metal, glass, plastic, polymers and ceramics.

In many embodiments, the outer chamber is compatible with sterilization techniques selected from the group consisting of ethylene oxide sterilization, gamma radiation sterilization, and e-beam radiation sterilization.

In many embodiments, the outer chamber includes a transparent portion for visualization of the medical implant container within.

In many embodiments, the kit further comprising a mechanical insert configured to engage the medical implant container and reduce stress imparted on the medical implant container when under the second vacuum.

In many embodiments, the mechanical insert is a rigid material selected from the group consisting of rigid polymer plastics, polystyrene, polypropylene, metal, acrylonitrile butadiene styrene and ABS plastic.

In many embodiments, medical implant is a medical graft.

Other objects, features and advantages of the present invention will become apparent to those skilled in the art from the following detailed description.

### BRIEF DESCRIPTION OF THE DRAWINGS

FIG. 1A is a top perspective view of an embodiment of a medical implant container.

FIG. 1B is a top perspective view of the medical implant container of FIG. 1 without the lid.

FIG. 1C is a cross-sectional view of the medical implant container of FIG. 1 A taken along the 1′-1′ axis

FIG. 2A is a front plan view of another embodiment of the medical implant container.

FIG. 2B is a rear perspective view of the medical implant container of FIG. 2A.

FIG. 3 is a top perspective view of a further embodiment of the medical implant container alongside a needle syringe.

FIG. 4A is a top perspective view of yet a further embodiment of the medical implant container.

FIG. 4B is a top perspective view of the medical implant container of FIG. 6A without the lid.

FIG. 5A is a top perspective view of yet a further embodiment of the of the medical implant container.

FIG. 5B is a top perspective view of the medical implant container of FIG. 5A showing substrate reconstitution using a syringe.

FIG. 6 is a data graph showing the relative binding strength of rhBMP-2 applied to Absorbable Collagen Sponges (ACS) contained in vacuum sealed package.

FIG. 7 is a data graph showing the relative binding strength of rhBMP-2 applied to allograft bone tissue contained in vacuum sealed package.

FIG. 8 is a top view of a container disposed within an outer chamber, wherein the outer chamber is under vacuum.

FIG. 9A-9D are various views of some embodiments of a mechanical insert for use with a suitably shaped medical implant container such as the container shown in FIGS. 1A-C and 5A-B.

FIG. 10 is a data graph showing the vacuum loss of a container housed under ambient film packaging.

FIG. 11 is a data graph showing the vacuum loss of a container housed under ambient foil packaging.

FIG. 12 is a data graph comparing the vacuum loss of containers housed in different outer chambers under vacuum over time.

FIG. 13 is a data graph showing the accelerated vacuum retention observed with various packaging materials at 55 degrees C.

Like numerals refer to like parts throughout the several views of the drawings.

### DETAILED DESCRIPTION OF EMBODIMENTS OF THE INVENTION

The present disclosure is directed to medical graft storage systems, kits for storing medical grafts under vacuum, and of increasing the shelf life of vacuum imposed on a medical implant prior to delivery to a patient. Although the present disclosure describes the methods and systems for medical grafts, particularly bone grafts, it is understood that the methods and systems can also be applied for a wide variety of medical and dental applications and also soft tissue applications, such as in regenerative medicine and tissue engineering. Accordingly, the term “graft” as used herein can be comprised of any naturally occurring tissue including bone tissue and soft tissues as well as any non-naturally occurring substance used as a graft, or any combination thereof.

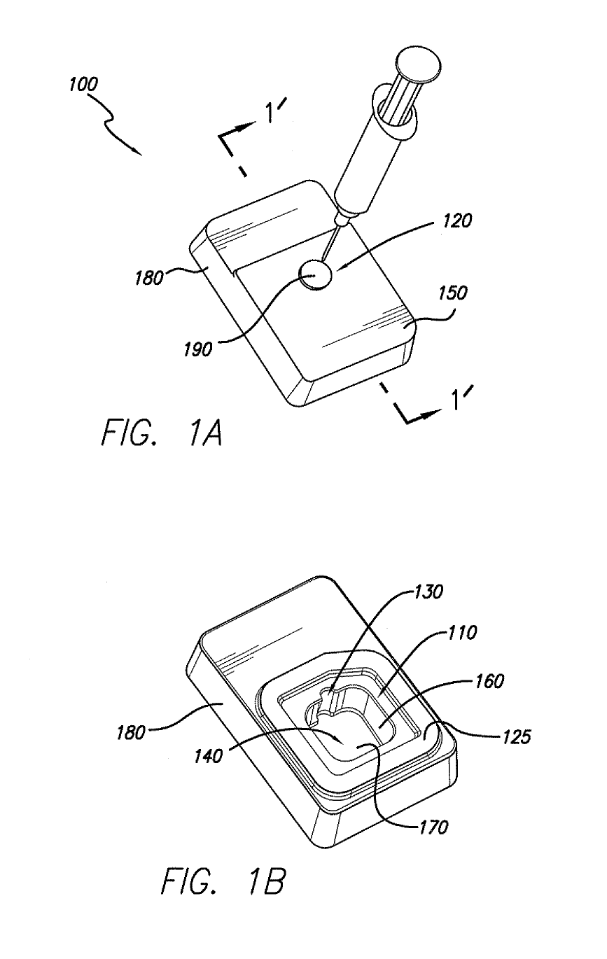

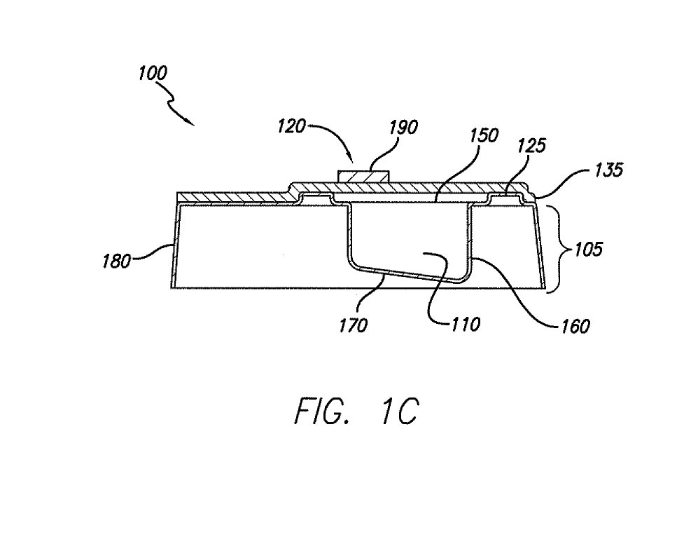

FIGS. 1A-C depict various views of an embodiment of a medical implant system 100 that can be used in connection with the systems and methods disclosed herein. As shown in FIGS. 1A-C, the medical implant system 100 comprises container 110 that includes an entry port 120, a needle cavity 130 and a graft cavity 140 containing a medical implant, such as a dehydrated bone graft (not shown). As shown in FIG. 1A, a needle syringe can be inserted through the entry port 120 to deliver liquids, biological components, and/or cells into the needle cavity 130 and to the graft that is stored in the graft cavity 140 of the container 110. The needle cavity 130 is disposed adjacent the graft cavity 140 to receive the needle syringe and the liquids, biological components and/or cell.

It is desirable to maintain the entire container 110 under vacuum and, more preferably, under substantial vacuum. This is because medical implants, such as bone grafts, are commonly dehydrated and freeze-dried for storage prior to use or implantation. The higher the negative pressure or vacuum within the container, the greater the evacuation of the pores within the graft and thus the greater infusion of the hydrating solution into the graft. Thus, it is preferable to have an absolute pressure inside the container as close to 0 mbar as possible. In a preferred embodiment, the absolute pressure inside the container is under 100 mbar, more preferably 10 mbar, and most preferably 1-5 mbar.

Freeze-drying involves a freezing process under negative pressure that results in a graft having low residual moisture. One advantage of this process is that it allows for storage of bone grafts and other biological material at room temperature. It also provides for increased shelf-life with reduced biochemical changes to the bone graft. Freeze-dried grafts thus offer the advantage of providing easy and economical storage prior to use.

In addition, it is preferable to reduce, if not completely eliminate, any residual moisture within the graft prior to packaging it in the container. This is because the negative pressure or vacuum in the container can cause the residual moisture to vaporize which, in turn, may cause the negative pressure or vacuum to decrease within the container. Preferably, the residual moisture within the graft is less than 6%, more preferably less than 3%, and most preferably 0%. A desiccant can be included in the container. The desiccant is preferably non-reactive with the graft or the solution that is used to hydrate the graft.

The bone graft is typically rehydrated or reconstituted with a saline solution prior to implantation in a patient or recipient. Rehydration of freeze-dried bone grafts typically involves soaking the grafts in the saline solution until the grafts reach the desired level of hydration. Depending on the size of the graft, among other factors, rehydration and reconstitution of a bone graft can take anywhere from one hour to a few days. Although it is desirable to achieve uniform penetration of the solution and homogenous rehydration of bone grafts, it is generally difficult to achieve these goals in the short period of time typically demanded in surgical environments.

The medical implant containers disclosed and described herein provide a means by which bone grafts, which have been freeze-dried or otherwise dehydrated, can be expeditiously and uniformly hydrated and reconstituted prior to implantation. Because the medical implant containers substantially maintain the vacuum during the hydration/reconstitution of the graft, the time for hydration or reconstitution is substantially reduced. The penetration of solution into the implant is enhanced by the vacuum induced suction effect. The vacuum produces a pressure differential that pushes the solution into the interstice or pores of the implant. Once the solution is distributed into the pores, it can be further distributed throughout the implant via capillary action.

In some embodiments, the medical implant vacuum infused package container system provides substantial hydration and reconstitution, along with substantially uniform seeding and loading of biological components and cells, within one minute to one hour from infusion.

In FIGS. 1A-C, since the entire container 110 is maintained under negative pressure or vacuum, it is desirable to reduce the internal volume of the container 110 to the extent necessary to house the bone graft. This is because it is generally more difficult to maintain negative pressure or vacuum for larger volumes of space. In a preferred embodiment, the volume of the container is substantially the same as the volume of the graft. In accordance with one aspect of this preferred embodiment, the volume of the container is no greater than approximately 125%, preferably no greater than approximately 110%, and more preferably no greater than 105%, of the volume of the graft. In accordance with another aspect of this preferred embodiment, the volume of the container is equal to the volume of the graft. As shown in FIGS. 1 A-C, the interior volume of the container 110 is bounded by top 150, side 160 and bottom 170 walls. A septum 190 is coupled to the entry port 120 and disposed externally of the container 110 so as to reduce an internal volume of the container 110 required to accommodate the septum. The materials selected for the container are preferably characterized as having high gas barrier properties to ensure that the negative pressure or vacuum is effectively maintained over time. In a preferred embodiment, the container is made from 40 gauge PETG (Pacur™ 6763).

The medical implant system 100 further comprises support members 180 to support the container 110 in a substantially stable and upright position. This will permit the surgeon to place the system 100 on a flat surface and simply insert a needle syringe into the entry port 120 with a single hand without having to support the system 100 with the other hand in the desired upright position. Although FIGS. 1A-C show the support members 180 as a single peripheral wall that surrounds the container 110, it is understood that the structure of the support members 180 is not so limited and can include other structures capable of stabilizing the container 110 in a sufficiently stable position to permit the surgeon to perform the injection step into the entry port 120.

The system 100 is shown to generally comprise a bottom portion 105 and a lid portion 135. The bottom portion 105 and the lid portion 135 can be hermetically-sealed by welding the two portions together so that vacuum can be maintained inside the container 110. It is preferable to position the weld as close to the periphery of the container 110 so as to further reduce the amount of dead airspace that may remain between the bottom portion 105 and the lid portion 135. The resulting weld 125 can surround the entire periphery of container 110. Although the system 100 depicted in FIGS. 1A-C is shown as a two-part structure comprising a bottom portion 105 and a lid 135, it is understood that the container can be constructed as an integral structure, such as an elastic vacuum package.

In addition to expeditious and uniform hydrating or reconstituting bone grafts, the system 100 promotes the efficient and uniform distribution and seeding of biological components and cells into the pores of the grafts. Biological components and cells can be delivered to the grafts in solution via needle syringe having the appropriate gauge so as to ensure against structural or cellular damage as they are passed through the needle syringe.

The interior surface of the container 110 is preferably configured to help preserve the integrity of the biological components and the cells during delivery to the bone graft. Particularly, the needle cavity 130 and the side 160 and bottom 170 walls are configured to promote a laminar flow of the biological solution received through the entry port. A laminar flow is characterized either as smooth or non-turbulent fluid flow. It is preferable to promote a laminar flow, and therefore reduce a turbulent flow, of the biological solution in the container 110 so as to preserve the structural and cellular integrity of the biological components and cells contained in the solution. A turbulent flow can, for example, cause the cells to become lysed and clump together. Eliminating, or at least reducing, sharp edges, corners or angles within the container 110 which the biological solution can come into contact with in the container can help promote a laminar flow of the solution. It is noted that because the liquid is expelled into the needle cavity and towards the bottom surface 170 of the container 110, the configuration of the top wall or lid portion 105 of the container 110 or where the side walls 160 meet the lid portion 105 of the container 110 are not as critical and therefore do not necessarily need to be curved.

As can be seen in FIGS. 1B-C, the side 160 and bottom 170 walls of the container 110 converge together as curved surfaces having radii of curvature greater than zero. Moreover, the internal surface of the needle cavity 130 is also provided as a curved surface having a radius of curvature greater than zero. Additionally, as depicted in FIG. 1C, the bottom wall 170 can be angled downward from the entry port 120 and the needle cavity 130 so as to ensure that the biological solution flows across and is distributed along a bottom length of the graft. This not only ensures the uniform distribution of the solution throughout the graft, it also prevents the pooling and waste of the solution in the needle cavity 130. Thus, embodiments of the medical implant container further provide for substantially precise dosing of a quantity of biological components or cells to be introduced. However, it is to be understood that in other embodiments, the bottom wall 170 need not be angled downward from the entry port 120 and the needle cavity 130.

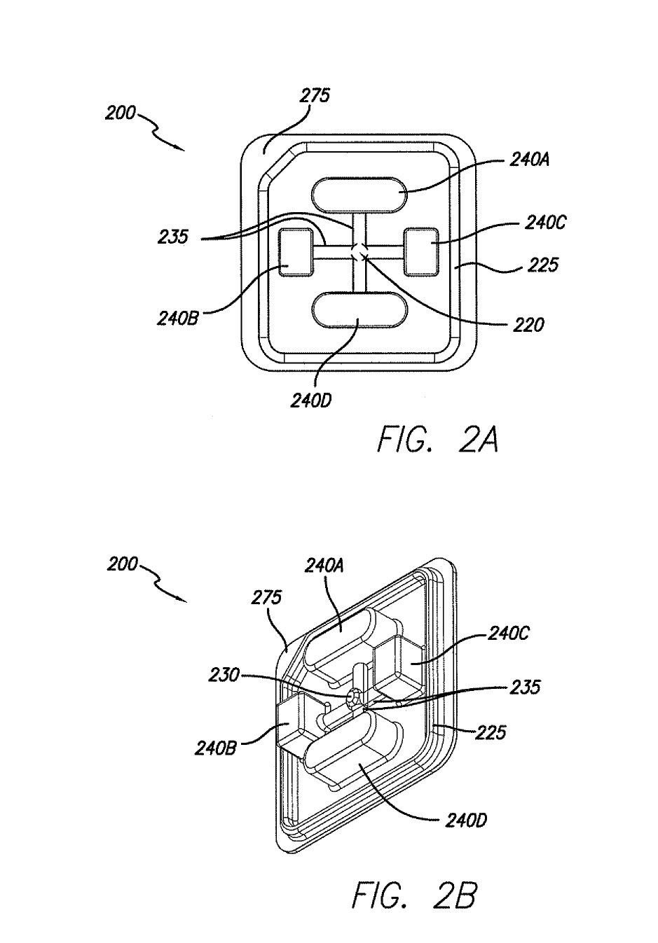

Alternate embodiments provide an efficient way to hydrate or reconstitute more than a single bone graft at the same time. FIGS. 2A-B show an embodiment of the medical graft container 200 comprising a single entry port 220 and a corresponding needle cavity 230 and a plurality of graft cavities 240A, 240B, 240C, and 240D coupled to and in fluid communication with the needle cavity 230 via channels 235. Once the grafts are loaded into the graft cavities 240A, 240B, 240C, and 240D, a gas communication is applied to the container so as to evacuate air remaining in the graft cavities. The container 200 can comprise a bottom portion and a lid that is optionally hermetically-sealed by a weld 225. A peripheral lip area 275 can be provided wherein the lid portion can be pulled apart from the bottom portion to open the container 200 and remove the bone grafts.

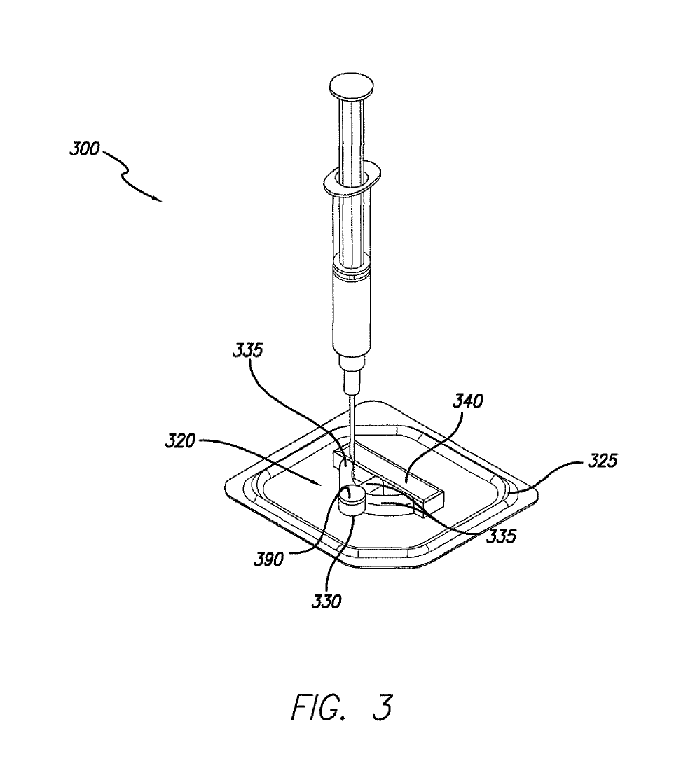

FIG. 3 depicts yet another embodiment of the medical graft container 300 alongside a needle syringe. The container 300 is designed to disperse the distribution of desired biological components and cells by providing a plurality of delivery channels 335 along a length of the graft cavity 340. The graft cavity 340 is preferably molded to the precise dimensions and shape of the dehydrated bone graft. The biological components and cells can be delivered via needle syringe through entry port 320 and into the needle cavity 330. A negative pressure or vacuum is maintained in the needle cavity 330, the delivery channels 335, and graft cavity 340. A septum 390 can optionally be coupled to the entry port 320 to maintain the negative pressure or vacuum after puncture with the needle syringe. The container 300 can be hermetically sealed by a weld 325 peripherally of the needle cavity 330, delivery channels 335 and graft cavity 340.

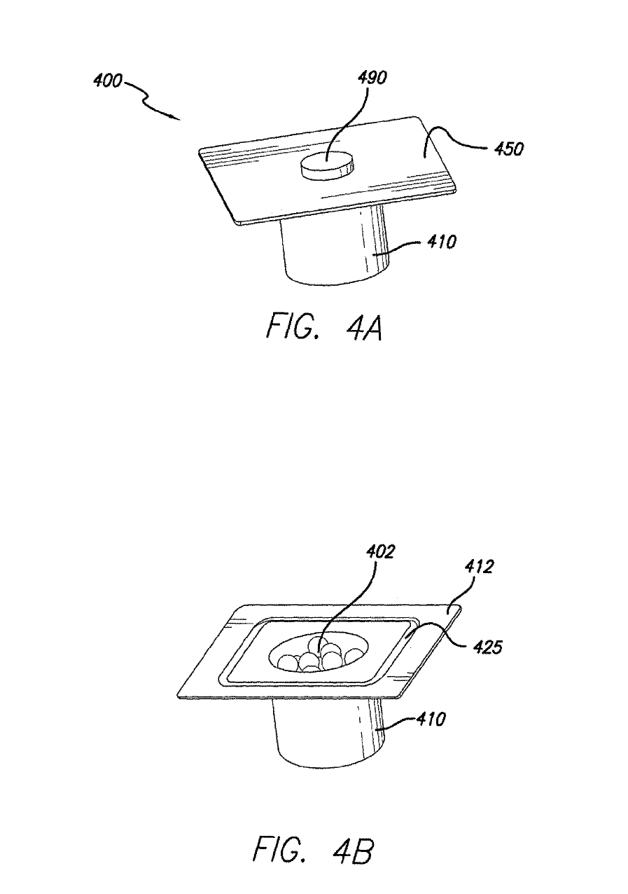

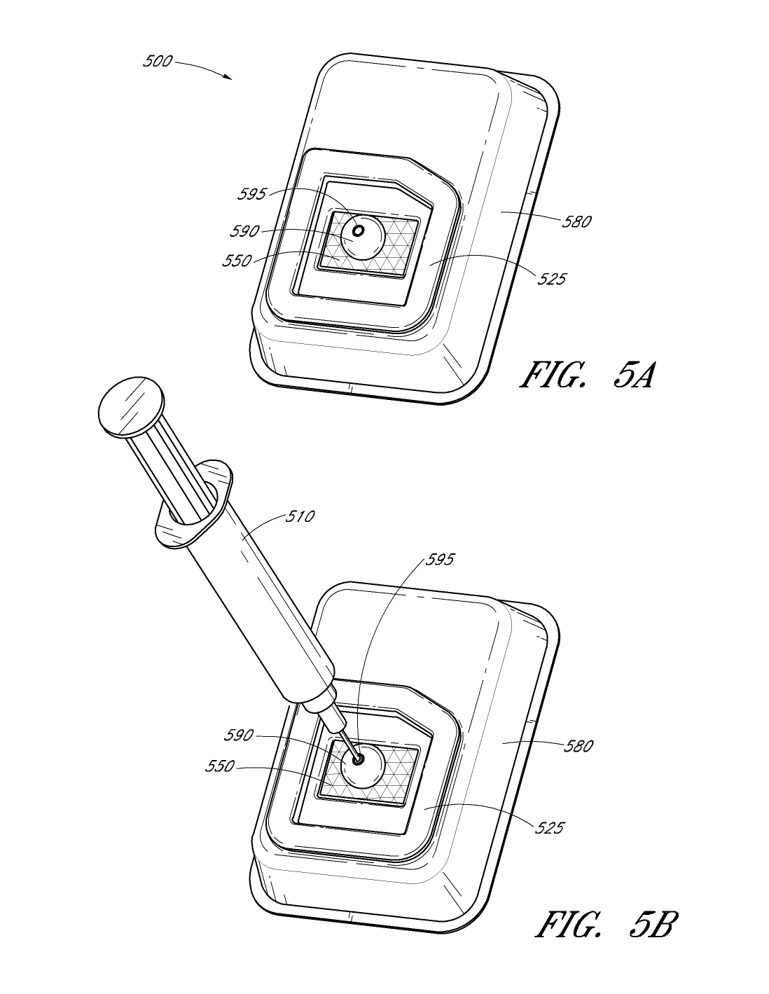

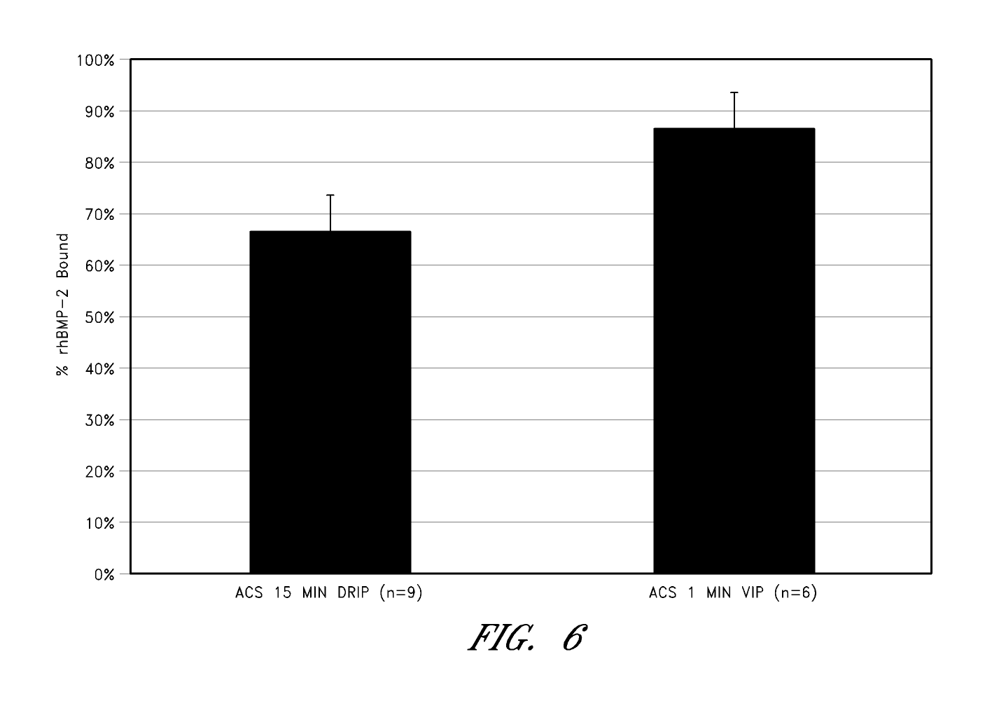

Other embodiments of the medical implant containers can be designed to reduce the internal volume that is maintained under vacuum. For example, FIGS. 4A-B depicts a medical implant container 400 comprising bone graft chips 402 contained within a graft cavity 410. A lid 450 is hermetically sealed to the peripheral lip 412 of the graft cavity 410 by a heat weld 425. As can be seen in FIGS. 4A-B, the bone graft chips 402 fill the graft cavity 410 to near capacity such that the volume of the graft cavity 410 is substantially the same as the volume of the bone graft chips 402. A septum 490 is disposed externally of the graft cavity 410. Preferably, the septum is self-sealing after puncture with a needle syringe delivering the hydrating solution to the graft chips 402 so as to sustain the vacuum inside the graft cavity 410.

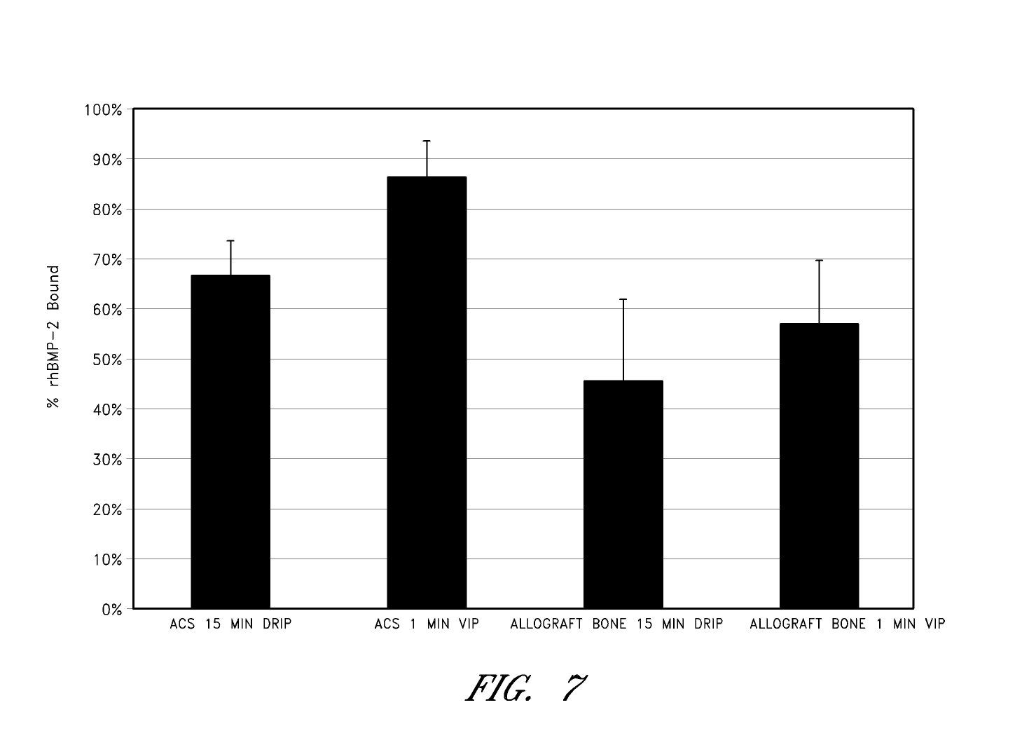

FIG. 5A depicts yet another embodiment of a medical graft container 500. FIG. 5B illustrates how a medical graft disposed within the medical graft container 500 of FIG. 5A can be infused with a suitable aqueous composition utilizing needle syringe 510. The container 500 is designed to disperse the distribution of desired biological components and cells from a distance closer to the middle of the medical graft by providing a septum 590 above the medical graft disposed within the graft cavity. The graft cavity is sealed under vacuum by a top web 550. This can be especially desirable for medical grafts that resist diffusion of the aqueous composition due to small pore size or other factors influencing the diffusion characteristics of the medical graft, such as viscosity of the aqueous composition. The medical graft container 500 can also be used in cases where it is desirable to set up a concentration gradient of the biological components or cells within a medical graft having a higher concentration of biological components and cells closer to the middle of the medical graft and a lower concentration of biological components and cells further away from the middle of the medical graft. The needle syringe 510 is inserted into the medical graft during infusion of the aqueous composition, or the needle syringe is merely placed above or adjacent to the medical graft during infusion. The graft cavity is preferably molded to the precise dimensions and shape of the medical graft. The biological components and cells can be delivered via needle syringe through entry port 595 of septum 590 to the medical graft disposed within the graft cavity. The container 500 can be hermetically sealed by a weld 525 peripheral to the graft cavity and support members 580 can be provided to support the container 500 in a substantially stable and upright position.

The aqueous compositions used herein to hydrate or reconstitute the bone grafts prior to implantation can be solutions, emulsions, micro-emulsions, suspensions or combinations thereof. Materials that function as emulsifiers or suspension aids can also be present in such aqueous compositions

In some embodiments, the aqueous compositions further contain water-miscible biocompatible solvents or solvent mixtures. The biocompatible solvents are preferably organic liquids in which the grafts are at least partly soluble at mammalian body temperatures and are substantially non-toxic in the quantities used.

Biological components used in connection with the medical implants disclosed herein include any agent that produces a biological, therapeutic or pharmacological result in a human. One group of biological components that are particularly useful in conjunction with bone grafts are Bone Morphogenetic Proteins (BMPs). BMPs are a group of growth factors and cytokines known for their ability to induce the formation of bone and cartilage. Examples of using BMPs in bone grafts is described in US Patent Application Publication No. 2004/0230310 Al entitled, “USE OF MORPHOGENETIC PROTEINS TO TREAT HUMAN DISC DISEASE,” which is herein incorporated by reference in its entirety.

The methods and systems disclosed herein can also be utilized to deliver living cells to desired sites in a recipient. These cells can be concentrated prior to implantation by methods such as centrifugation or filtration. Thus, the medical implants seeded can function as adhesion substrates, anchoring cells to be transplanted to effect the survival, growth and ultimately, grafting or anchoring of the transplanted cells to normal cellular tissue.

Porous substrates which can be used in connection with the disclosed methods and systems include autograft, allograft, xenograft, or other non-human animal-based materials such as collagen and other peptide comprising implants. Synthetic materials including ceramics, hydroxyapatite, bioresorbable polymers and the like can also be used as graft materials. In some embodiments, the porous substrate is an osteoconductive matrix comprising a biologically acceptable matrix sponge. The sponge is preferably a collagen sponge as will be described in greater detail below.

In some embodiments, the synthetic substrates include polymers that are biostable, while in other embodiments, the synthetic substrates include polymers that are bioresorbable.

In some embodiments, the porous substrate is a bioabsorbable absorbent matrix. One example of a suitable absorbent matrix is an Absorbable Collagen Sponge (ACS) as is taught in U.S. Patent Application Publication No. 2007/0142916 A1 entitled “BONE GRAFT COMPOSITION, METHOD AND IMPLANT,” which is herein incorporated by reference in its entirety.

In some embodiments, the absorbent matrix is derived from Type I bovine tendon collagen. The collagen matrix preferably has pores of a sufficient size and quantity to permit growing tissue to infiltrate therein. The collagen matrix can also comprise a multiplicity of substantially rigid nanofibers dispersed within the collagen matrix to impart structural integrity to the collagen matrix with nanofiber ends projecting out of a surface of the collagen matrix to provide differential load bearing surface bristles.

“Nanofiber” includes such structures as nanowires, nanowhiskers, semi-conducting nanofibers, carbon nanotubes and composite nanotubes so long as they impart a bristled surface to the resorbable osteoconductive matrix of the invention.

Although collagen is a good example of a rigid nanofiber, other polymers are suitable as well. Derivatives of other biopolymers that are rod-like, such as tubulin and keratin that can be manufactured in rigid nanofiber form can be suitable so long as they retain a fiber structure integrity under conditions of matrix formation. A preferred nanofiber is a nanometer scale rod-like polymer that is water compatible and has polar surface groups such as amino groups.

Other embodiments of the present invention include use of the vacuum package container disclosed herein in conjunction with an INFUSE® Bone Graft device (Medtronic Sofamor Danek, Memphis, Tenn.) and can include a Bone Graft/LT-CAGE® Lumbar Tapered Fusion Device (Medtronic Sofamor Danek, Memphis, Tenn.) disposed within the Vacuum Infused Package (VIP) medical container. The INFUSE® device comprises two parts: (1) a genetically-engineered human protein (rhBMP-2) to stimulate bone healing, and; (2) an absorbable collagen sponge scaffold made from cow (bovine) collagen that carries the BMP, as described above.

In yet further embodiments, the vacuum packaging can also contain other items disposed within it such as mechanical devices including metal plates, pins, rods, wires, screws, and Graft/LT-CAGE's® or any other suitable structural element either singularly or in combination with a porous substrate.

Freeze-dried (lyophilized) porous substrate, such as an absorbent matrix, can be difficult to hydrate and is often ineffectually hydrated in the operating room (OR) due to the amount of time it takes to hydrate the porous substrate using conventional “soaking” methods. The medical container disclosed herein embodies a novel method for rapidly rehydrating a porous substrate, decreasing the brittleness of the substrate, and delivering biological components and cells to the porous substrate in an effective and efficient manner. The container seals a dehydrated porous substrate under an extremely strong vacuum by evacuating the air from the pores of the substrate. During fluid infusion, the vacuum pulls the fluid into the porous substrate, rapidly infusing the pores and rehydrating the implant.

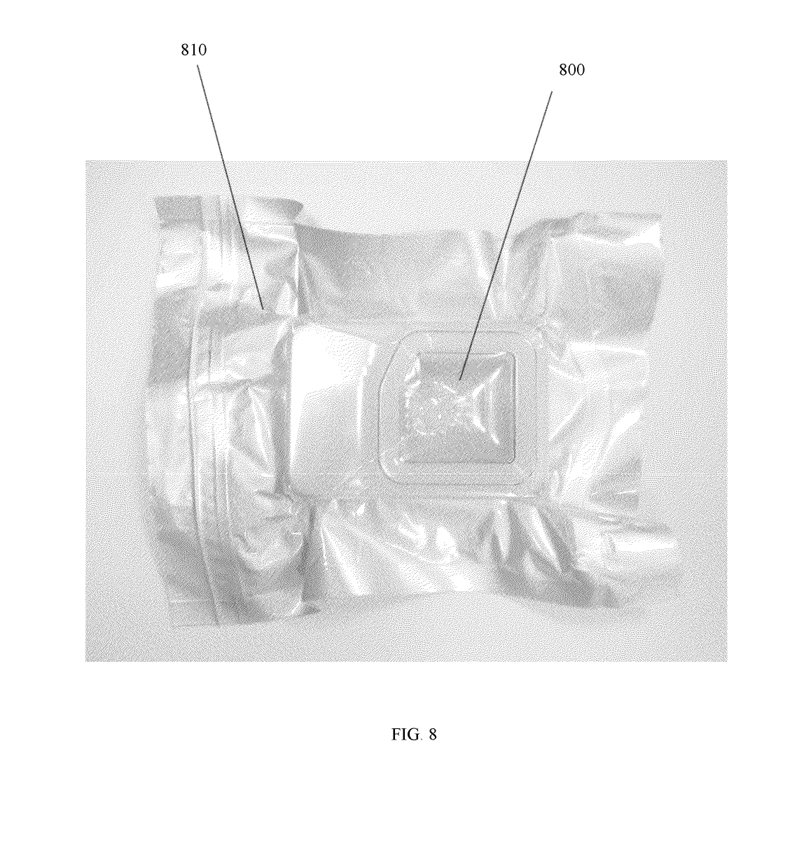

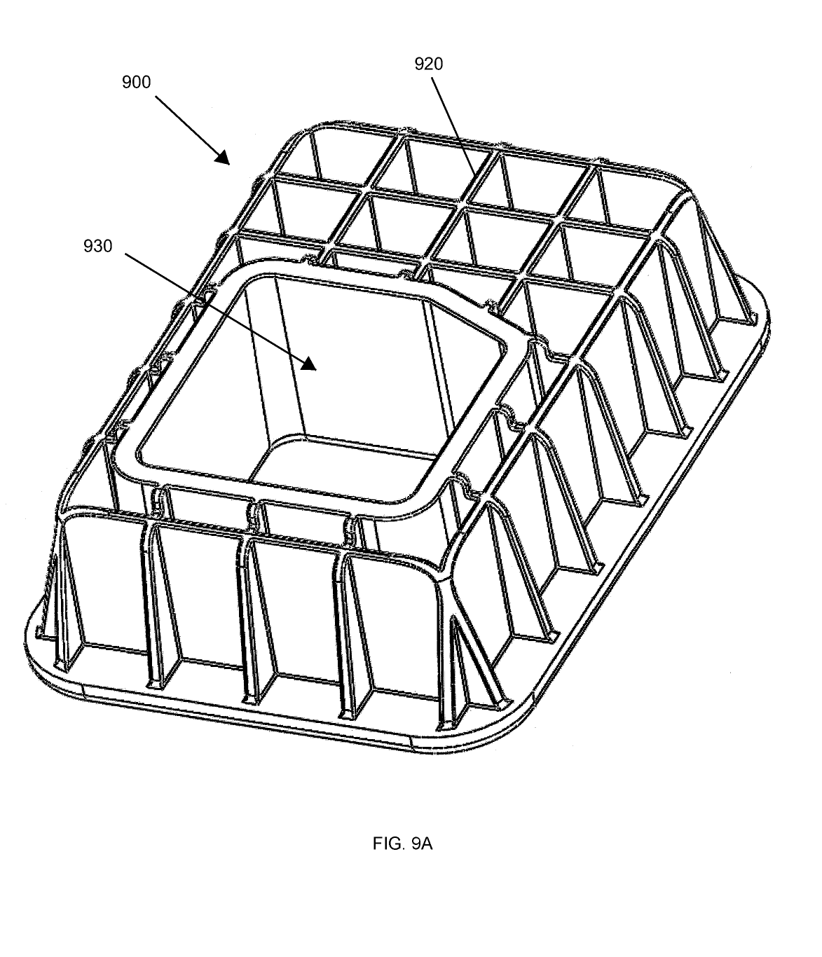

FIG. 8 shows an embodiment of a kit or system having a medical graft container under vacuum, as described above, disposed within an outer chamber which is also under vacuum, in either. Vacuum is applied to the outer chamber such that both the graft container and the outer chamber are under vacuum. In some embodiments, the graft container and the outer chamber are under substantially the same vacuum. In other embodiments, the graft container and the outer chamber are under different vacuums. For example the graft container may be under a first vacuum and the outer chamber may be under a second vacuum.

In one embodiment, the graft container 800 is sealed under vacuum of about 10 inHg. Other embodiments have graft containers sealed under vacuum of between about 1 to about 30 inHg. Other suitable vacuums are also contemplated. Graft container 800 can be sealed using a top web comprising, for example, a Perfecseal® film (31868-G) top web or a Tolas® foil film (TCP-0184B) top web and vacuum sealer (not shown). Perfecseal® medical packaging products and specifications can be found at: http://www.perfecseal.com, and Tolas® healthcare packaging products and specifications can be found at: http://www.tolas.com. The vacuum sealed graft container 800 can then be placed in an outer chamber 810, such as a SiOx, foil or Alox film pouch and vacuum is then applied to the outer chamber 810 using a chamber pouch sealer (not shown) such that both the graft container 800 and the outer chamber 810 are under vacuum. In one embodiment, the outer chamber 810 is sealed under vacuum of about 10 inHg. Other embodiments have outer chambers 810 sealed under vacuum of between about 1 to about 30 inHg. Other suitable vacuums are also contemplated.

The outer chamber is preferably comprised of a high barrier film to prevent vacuum loss. As will be appreciated by a person having ordinary skill in the art, any film which possesses the quality of serving as a barrier to oxygen, water or other atmospheric substances can be utilized. Suitable films include, for example, SiOx, foil, ACLAR, EVOH, PVOH. Alox , SiOx-F and PET. In some embodiments, Rollprint® packaging products, such as RPP# 37-1021A, can be used for the outer chamber. In other embodiments, Pacur™ packaging products, such as Pacur™ 6763 Copolyester Sheet, can be used for the outer chamber.

In other embodiments, the outer chamber can be made of one or more rigid materials suitable for holding vacuum including, but not limited to: metal, glass, plastic, polymers, ceramics, etc. In these embodiments, a chamber tray sealer or other vacuum chamber sealer (not shown) can be used to apply vacuum to the more rigid outer chambers of these embodiments.

The outer chamber 810 is constructed of materials compatible with standard sterilization techniques including, for example, ethylene oxide sterilization, gamma radiation sterilization, and e-beam radiation sterilization. In some embodiments, the outer chamber is constructed as a pouch. In some embodiments, the outer chamber includes at least one transparent side or portion to allow for visualization of the graft container within.

The inclusion of an outer chamber provides many advantages over the prior art. These advantages include, without limitation, longer shelf-life due to more reliable and sustained vacuum imposed on the medical device, an improved sterility barrier, and increased moisture barrier. In some embodiments, the outer chamber helps maintain packaging integrity with respect to vacuum retention, moisture protection, and sterility for at least 3 years. Another advantage of the use of an outer container in connection with the medical graft container is the improved and sustained vacuum that is achieved, resulting in an increased rate and quality of rehydration of the graft material.

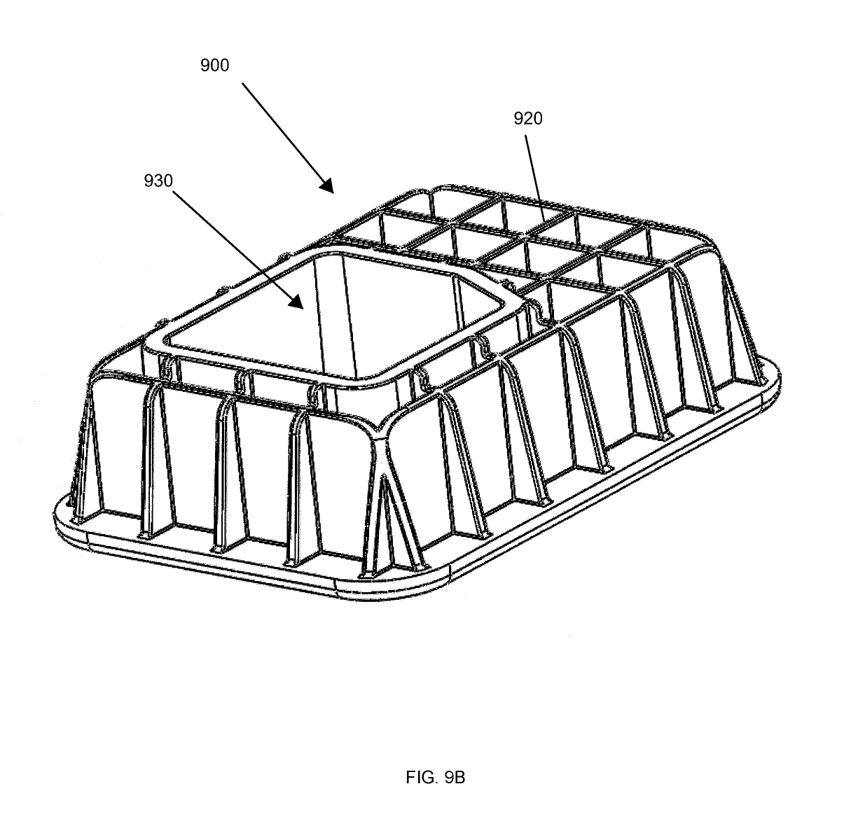

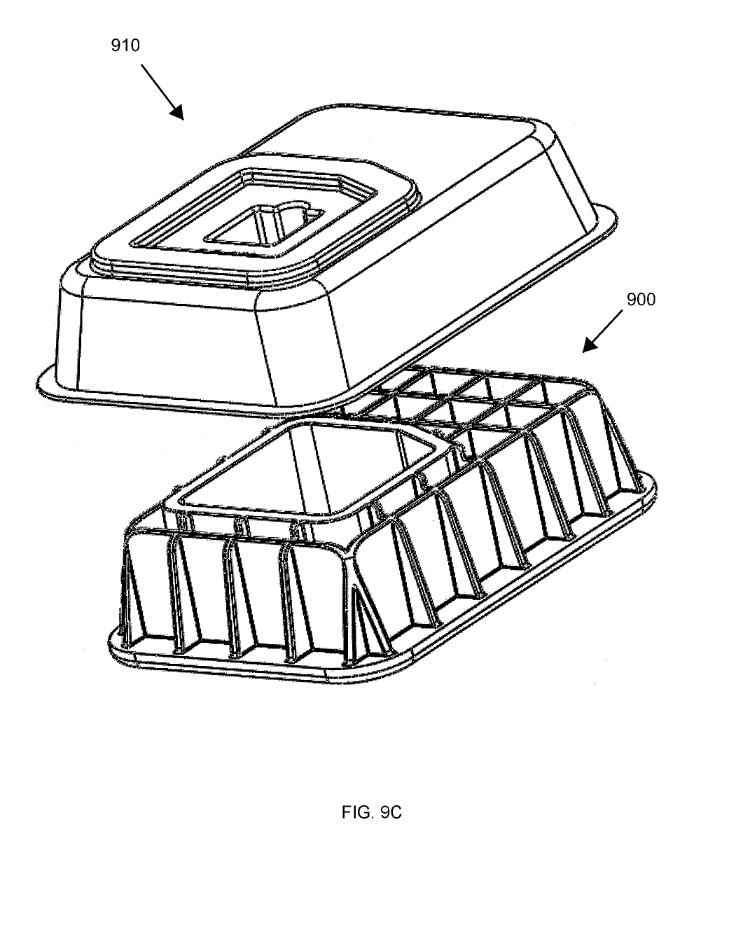

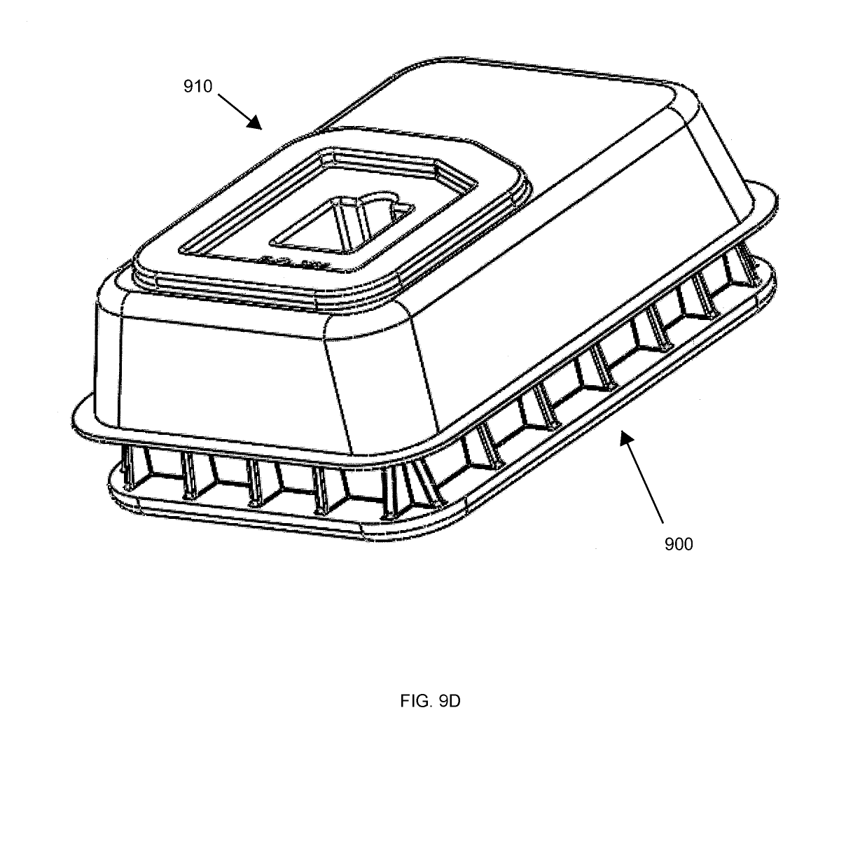

FIGS. 9A-9D show one embodiment of a mechanical insert for use with a medical graft container, as described above. The insert 900 includes a plurality of rigid support ribs 920. However, it will be appreciated by one of skill in the art that any rigid material in any suitable configuration can also be used, wherein the insert provides a platform for the medical graft container to sit. The insert 900 preferably has a graft cavity chamber 930 suitably shaped for receiving the graft cavity portion of a similarly shaped medical graft container.

Referring now to FIGS. 9C and 9D, the insert 900 is configured such that a medical graft container 910 can be seated over the mechanical insert 900 providing support to the medical graft container 910 when vacuum is applied by an outer container, such as outer container 810. Thus, the insert 900 provides structural support such that the amount of stress imparted to the medical graft container/medical graft, is reduced when vacuum is applied. The insert 900 is constructed from any relatively rigid material which is capable of withstanding vacuum. Some examples of suitable materials include, without limitation, relatively rigid polymer plastics such as polystyrene, polypropylene, other standard plastics, metals, and acrylonitrile butadiene styrene or ABS plastic.

### Experiments

The following examples teach medical implants and methods and systems for hydrating and seeding medical implants with biological components. These examples are illustrative only and are not intended to limit the scope of the invention disclosed herein. The treatment methods described below can be optimized using empirical techniques well known to those of ordinary skill in the art. Moreover, artisans of skill would be able to use the teachings described in the following examples to practice the full scope of the invention disclosed herein.

### Experiment 1

An experiment was conducted using an aqueous solution containing a known concentration of rhBMP-2 and multiple ACS sponges. 150 μg of rhBMP-2 (Infuse®, Medtronic, Inc., Memphis, Tenn.) per cc of carrier was delivered into absorbable collagen sponges (ACS, Medtronic, Inc.), using either a drip (soaking) method or via Vacuum Infused Packaging (VIP). The rhBMP-2 applied by the drip method was allowed to soak for 15 minutes while the VIP samples were only allowed 1 minute for binding. Unbound rhBMP-2 was rinsed out of the ACS sponges by being placed in excess saline on an orbital shaker at 37° C. for 1 hour. An rhBMP-2 ELISA kit (Leinco Technologies, Inc., St. Louis, Mo.) was used to determine the amount of rhBMP-2 that was bound to the ACS samples after the 1 hour rinse. Data were then analyzed using a one-way ANOVA (p<0.05) and Tukey's post-hoc honest significant difference test for multiple comparisons.

The amount of rhBMP-2 bound to the dripped ACS after 15 minutes of binding time versus ACS after 1 minute VIP infusion time is shown in FIG. 6. Some ACS sponges were soaked in rhBMP-2 solution (DRIP) and other ACS sponges were infused (using the same rhBMP-2 solution) inside of vacuum sealed package. As can be seen in FIG. 6, the VIP infused ACS exhibited statistically greater binding of rhBMP-2 as compared to the 15 minute soaked (DRIP) ACS, even with a substantially lessened binding time of only 1 minute compared to the 15 minutes allotted to the soaked ACS (p<0.0035). These surprising and unexpected results indicate that VIP increases the binding ability of rhBMP-2 to ACS material. Without being bound to a particular theory, it is believed that the rhBMP-2 binds better to the ACS using VIP over traditional soaking methods because VIP facilitates greater binding by exposing the rhBMP-2 to a greater number of collagen binding sites within the ACS.

### Experiment 2

A similar experiment was performed using allograft bone tissue, instead of ACS sponges, to further verify that the foregoing “surprising and unexpected” results were not unique to ACS sponges. 150 μg of rhBMP-2 (Infuse®, Medtronic, Inc., Memphis, Tenn.) per cc of carrier was delivered to multiple allograft bone tissue samples using either a drip (soaking) method or via Vacuum Infused Packaging (VIP). The rhBMP-2 applied by the drip method was allowed to soak for 15 minutes while the VIP samples were only allowed 1 minute for binding. Unbound rhBMP-2 was rinsed out of the allograft bone tissue samples by placing them in excess saline on an orbital shaker at 37° C. for 1 hour. An rhBMP-2 ELISA kit (Leinco Technologies, Inc., St. Louis, Mo.) was used to determine the amount of rhBMP-2 that was bound to the allograft bone tissue samples after the 1 hour rinse. Data were then analyzed using a one-way ANOVA (p<0.05) and Tukey's post-hoc honest significant difference test for multiple comparisons.

FIG. 7 shows the relative amounts of rhBMP-2 bound to the dripped allograft bone tissue samples after 15 minutes of binding time versus bone samples having a 1 minute VIP infusion time. As is seen in FIG. 7, the VIP infused allograft bone samples exhibited statistically greater binding of rhBMP-2 as compared to the 15 minute soaked (DRIP) allograft bone samples, even with a substantially lessened binding time of only 1 minute compared to the 15 minutes allotted to the soaked bone samples. These surprising and unexpected results further indicate that VIP increases the binding ability of rhBMP-2 in multiple absorbent matrix materials, not just in ACS sponges.

In some embodiments, the medical implant vacuum infused package container system provides greater than 50% binding of biological components to a bioabsorbable absorbent matrix in less than 15 minutes from infusion. In other embodiments, the medical implant vacuum infused package container system provides greater than 50% binding of biological components to a bioabsorbable absorbent matrix in less than 14, 13, 12, 11, 10, 9, 8, 7, 6, 5, 4, 3 or 2 minutes from infusion, and preferably in less than or equal to 1 minute from infusion.

In other embodiments, the medical implant vacuum infused package container system provides greater than 75% binding of biological components to a bioabsorbable absorbent matrix in less than 15 minutes from infusion. In other embodiments, the medical implant vacuum infused package container system provides greater than 75% binding of biological components to a bioabsorbable absorbent matrix in less than 14, 13, 12, 11, 10, 9, 8, 7, 6, 5, 4, 3 or 2 minutes from infusion, and preferably in less than or equal to 1 minute from infusion.

The benefits of increased binding between rhBMP-2 and ACS in a VIP environment are immediate and identifiable. Strong binding of rhBMP-2 to ACS is very desirable because this lessens premature precipitation of rhBMP-2 out of absorbent matrix grafts and into surrounding tissue inside the patient's body. As discussed previously, premature or excessive precipitation of BMPs has been known to stimulate ectopic bone growth in muscle tissue and in more serious cases, involving implants in the cervical spinal area, ectopic bone growth has been known to completely surround the subject's trachea closing off their air passage and causing suffocation.

### Experiment 3

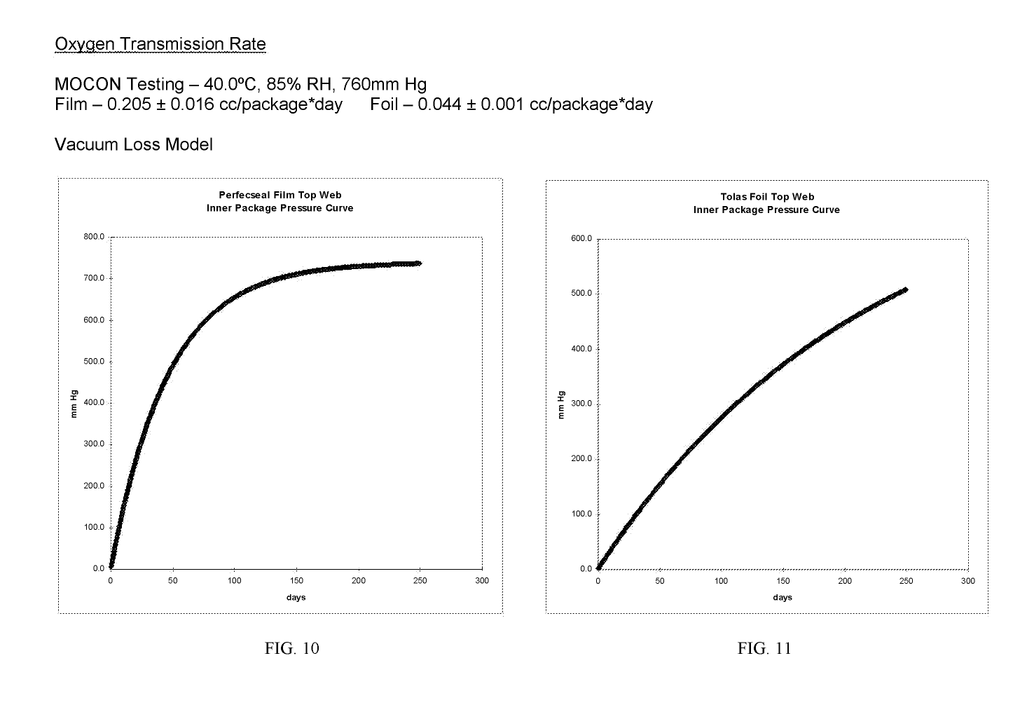

Referring to FIGS. 10 and 11, an experiment was performed comparing the vacuum loss of two medical graft containers under vacuum without any additional vacuum seal outer packaging. Accordingly, FIGS. 10 and 11 graphically depict the transmission of oxygen or vacuum loss in a container sealed with a top web only, where no additional vacuum seal outer packaging was utilized. One medical graft container was sealed with a Perfecseal® top web, while the other medical container was sealed with a Tolas® foil top web. The medical containers were then exposed to conditions of 40 degrees C., 85% RH, and 760 mm HG pressure. Vacuum loss was measured over a period of about 250 days. The results of the experiment are shown in FIGS. 10 and 11. Vacuum loss models revealed that the Perfecseal® film top web sealed container gained an average of 0.205±0.016 cc of air per day, whereas the Tolas® foil film top web sealed container gained an average of 0.044±0.001 cc of air per day. Accordingly, the Tolas® foil film top web was evidently better at holding vacuum than the Perfecseal® top web.

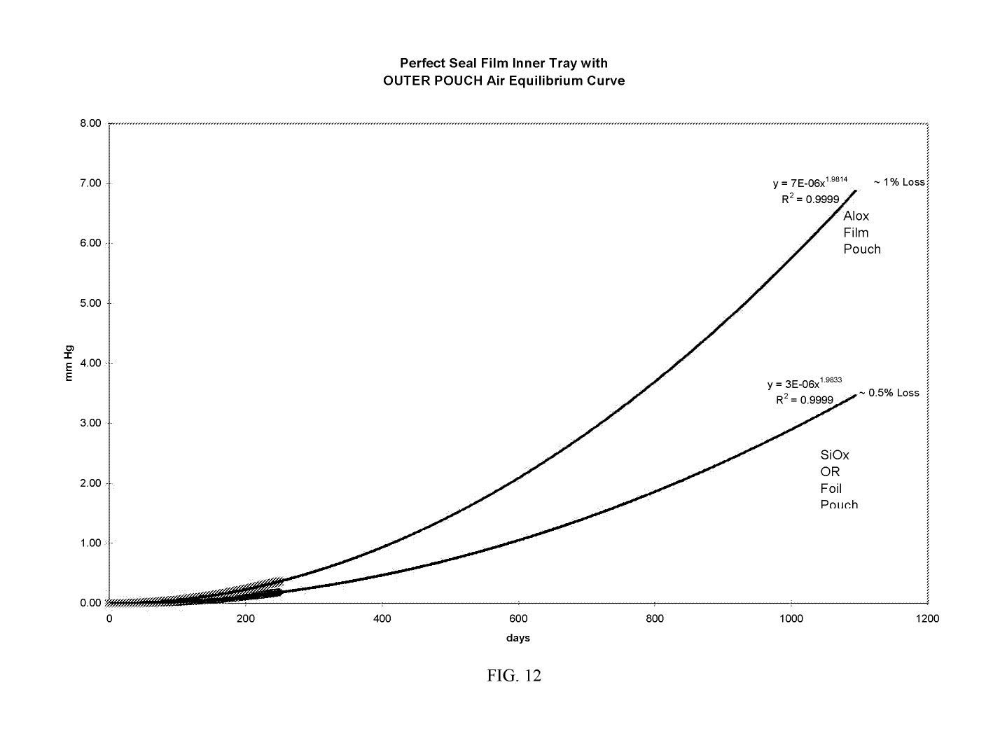

Referring now to FIG. 12, an experiment was performed comparing the vacuum loss of multiple medical graft containers which were first sealed under vacuum using Perfecseal® film top webs and then sealed again under vacuum in differing outer chambers. One outer chamber comprised an Alox film pouch and the other was a SiOx or foil pouch. The sealed medical graft containers were then stored at 55° C. and vacuum infusion was measured periodically by weighing the sealed medical graft containers. FIG. 12 illustrates the reduction of vacuum loss observed for the medical graft containers housed in the different outer chambers. The loss in vacuum was measured over a period of about 250 days and vacuum loss model was extrapolated from this data as can be seen in FIG. 12. The results of this experiment show generally that the addition of an outer vacuum chamber will lengthen vacuum life. More specifically, the results of this experiment show that different outer chamber materials can lengthen vacuum life better than others, depending on the specific material(s) used to construct the outer chamber.

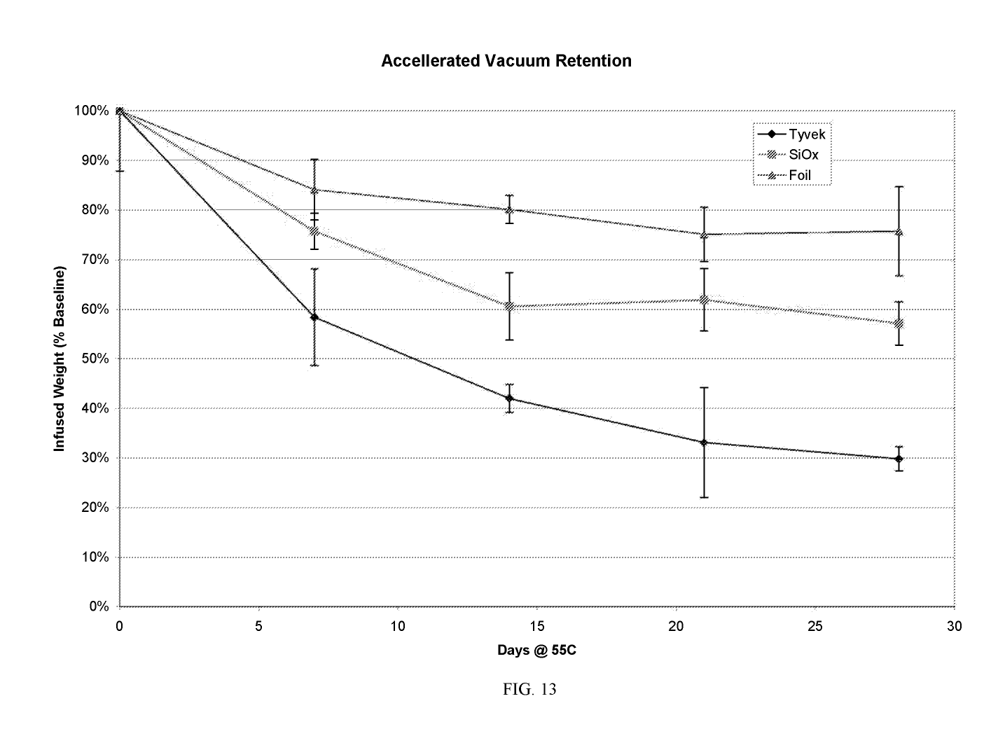

FIG. 13 illustrates the results of an experiment in a data graph showing the accelerated vacuum retention observed with various outer chamber packaging materials. An experiment was performed comparing the vacuum loss of three medical graft containers which were first sealed under vacuum using top webs and then sealed again under vacuum in three different outer chambers. One outer chamber was an Tyvek® film pouch, another was a SiOx pouch and the other was foil pouch. The vacuum sealed containers were then stored at 55° C. and their infused weight (as a percentage of baseline) was then measured over a period of about 30 days. FIG. 13 illustrates the reduction of vacuum loss observed for all of theses medical graft containers housed in their different outer chambers. More specifically, the results of this experiment show that different outer chamber materials can lengthen vacuum life better than others. For example, the container stored in a foil pouch outer chamber retained its vacuum better than the containers stored in the SiOx and Tyvek outer chambers.

It is to be understood that the detailed description and specific examples, while indicating preferred embodiments of the present invention, are given by way of illustration and not limitation. Many changes and modifications within the scope of the present invention can be made without departing from the spirit thereof, and the invention includes all such modifications.

1-20. (canceled)

21. A kit for storing freeze-dried or dehydrated medical grafts under vacuum, comprising:
a hermetically sealed medical implant container comprising:
at least one graft cavity configured to store at least one graft under a first sustained vacuum;
top, side, and bottom walls defining an inner surface of the container;
a lip surrounding a periphery of the graft cavity with the top wall hermetically sealed to the lip adjacent the substrate cavity periphery; and
a needle entry port in fluid communication with the at least one graft cavity, the needle entry port being configured to receive and communicate a material to the at least one graft cavity; and
an outer chamber configured to store the medical implant container under a second sustained vacuum.

22. The kit of claim 21, wherein the first vacuum and second vacuum are between 1 and 30 inHg.

23. The kit of claim 21, wherein the first vacuum and second vacuum are substantially the same.

24. The kit of claim 21, wherein the first vacuum and second vacuum are not substantially the same.

25. The kit of claim 21, wherein the outer chamber is made of a material selected from the group consisting of silicon oxide (SiOx), foil, ACLAR® polychlorotrifluoroethylene (PCTFE), polyvinyl alcohol (PVOH), aluminum oxide (Alox), Silicon Oxide-F (SiOx-F) and polyethylene terephthalate (PET), metal, glass, plastic, polymers and ceramics.

26. The kit of claim 21, wherein the outer chamber is compatible with sterilization techniques selected from the group consisting of ethylene oxide sterilization, gamma radiation sterilization and e-beam radiation sterilization.

27. The kit of claim 21, wherein the outer chamber includes a transparent portion for visualization of the medical implant container within.

28. The kit of claim 21, further comprising a mechanical insert configured to engage the medical implant container and reduce stress imparted on the medical implant container when under the second vacuum.

29. The kit of claim 28, wherein the mechanical insert is a rigid material selected from the group consisting of rigid polymer plastics, polystyrene, polypropylene, metal, and acrylonitrile butadiene styrene plastic.

30. A medical graft storage system, comprising:
at least one freeze-dried or dehydrated medical graft;
a hermetically sealed medical implant container comprising:
at least one graft cavity configured to store at least one graft under a first sustained vacuum;
top, side, and bottom walls defining an inner surface of the container;
a lip surrounding a periphery of the graft cavity with the top wall hermetically sealed to the lip adjacent the substrate cavity periphery; and
a needle entry port in fluid communication with the at least one graft cavity, the needle entry port being configured to receive and communicate a material to the at least one graft cavity; and
an outer chamber configured to store the medical implant container under a second sustained vacuum.

31. The system of claim 30, wherein the first vacuum and second vacuum are between 1 and 30 inHg.

32. The system of claim 30, wherein the first vacuum and second vacuum are substantially the same.

33. The system of claim 30, wherein the first vacuum and second vacuum are not substantially the same.

34. The system of claim 30, wherein the outer chamber includes a transparent portion for visualization of the medical implant container within.

35. The system of claim 30, wherein the outer chamber is made of a material selected from the group consisting of silicon oxide (SiOx), foil, ACLAR® polychlorotrifluoroethylene (PCTFE), polyvinyl alcohol (PVOH), aluminum oxide (Alox), Silicon Oxide-F (SiOx-F) and polyethylene terephthalate (PET), metal, glass, plastic, polymers and ceramics.

36. The system of claim 30, further comprising a mechanical insert configured to engage the medical implant container and reduce stress imparted on the medical implant container when under the second vacuum.

37. The system of claim 30, wherein the material is a biological material.

38. A system for loading bone morphogenetic material into a medical graft, comprising:
a hermetically sealed container comprising an entry port, a substrate cavity, and top, side and bottom walls defining an inner surface of the container, a lip surrounding a periphery of the substrate cavity and the top wall is hermetically sealed to the lip adjacent the substrate cavity periphery;
the entry port configured to receive the bone morphogenetic material;
the substrate cavity in communication with the entry port and having the medical graft stored and maintained under negative pressure,
wherein a container volume is substantially the same as a volume of the medical graft; and an outer chamber configured to store the medical implant container under a second sustained vacuum.

39. The system of claim 38, wherein the entry port is disposed near the middle of the medical graft within the substrate cavity

40. The system of claim 38, wherein the container further comprises one or more supporting members configured to elevate and support the container in a substantially stable position.
